(19) United States
(12) Patent Application Publication
(10) Pub. No.: US 2010/0331709 A1
(43) Pub. Date: Dec. 30, 2010

(54) DETECTION UNIT FOR BLOOD PRESSURE INFORMATION MEASUREMENT DEVICE AND BLOOD PRESSURE INFORMATION MEASUREMENT DEVICE

(75) Inventors: Naomi Matsumura, Kyoto (JP); Yukiya Sawanoi, Kyoto (JP); Kenji Fujii, Kyoto (JP); Reiji Fujita, Kyoto (JP)

Correspondence Address: OSHA LIANG L.L.P., TWO HOUSTON CENTER, 909 FANNIN, SUITE 3500, HOUSTON, TX, 77010, US

(73) Assignee: OMRON HEALTHCARE CO., LTD., Kyoto-shi, Kyoto (JP)

(21) Appl. No.: 12/866,160

(22) PCT Filed: Jan. 19, 2009
(86) PCT No.: PCT/JP2009/050652

§ 371 Date: Aug. 4, 2010

(30) Foreign Application Priority Data
Feb. 8, 2008 (JP) 2008-029322

(51) Int. Cl. A61B 5/022

(52) U.S. Cl. 600/490

(57) ABSTRACT

A blood pressure information measurement device includes a cuff (10A) serving as a detection unit. The cuff (10A) includes an air bladder (40), a photoelectric sensor (50), a belt member (20), and a fixing stand (32), and is attached to a living body by wrapping the belt member (20) around a measuring site. The photoelectric sensor (50) optically detects an intra-arterial volume fluctuation, and includes a light emitting element (51) and a light receiving element (52). The fixing stand (32) includes a base portion (32a) with a sensor attachment surface (32a1) attached with the photoelectric sensor (50), and a guide portion (32b) arranged projecting out from the base portion (32a) toward the sensor attachment surface (32a1) side, and having a distal end placed at a body surface near the measuring site in the attachment state of the cuff (10A). The air bladder (40) is arranged on the sensor attachment surface (32a1) so as to cover the photoelectric sensor (50), and the guide portion (32b) is arranged to surround the photoelectric sensor (50). With such a configuration, the blood pressure information measurement device capable of acquiring a volume pulse wave easily and at high accuracy can be obtained.

### TECHNICAL FIELD

The present invention relates to a blood pressure information measurement device for acquiring blood pressure information through optical methods, and a detection unit of the same.

### BACKGROUND ART

Acquiring blood pressure information of a subject is very important in terms of knowing a health condition of the subject. In recent years, it is not limited to acquiring a systolic blood pressure value, a diastolic blood pressure value, and the like, which effectiveness has been widely recognized as a representative index of health management from the conventional art, and attempts have been made in capturing a change in a heart load and hardness of an artery by acquiring a pulse wave of the subject. The blood pressure information measurement device is a device for obtaining the index for health management based on the acquired blood pressure information, and further utilization is expected in fields of early detection and prevention, treatment, and the like of circulatory system diseases. The blood pressure information widely includes various information of the circulatory system such as the systolic blood pressure value, the diastolic blood pressure value, an average blood pressure value, a pulse wave, a pulse beat, and an AI (Augmentation Index) value.

The pulse wave, which is one type of blood pressure information, includes a pressure pulse wave and a volume pulse wave due to a difference in a target to capture. In the pressure pulse wave, the pulse wave is captured as a fluctuation of an intravascular pressure involved in beating of the heart, and in the volume pulse wave, the pulse wave is captured as a fluctuation of a intravascular volume involved in the beating of the heart. The fluctuation of the intravascular volume is a phenomenon that occurs with the fluctuation of the intravascular pressure, and thus the pressure pulse wave and the volume pulse wave are assumed to be indices having substantially similar medical meaning. The fluctuation of the intravascular volume can be recognized as a fluctuation of blood tissue amount in a blood vessel.

The term blood pressure information measurement device used herein refers to the overall device having at least a function of acquiring the pulse wave, and more specifically, refers to a device for acquiring the volume pulse wave by detecting the fluctuation of the blood tissue amount through an optical method. In this regard, the blood pressure information measurement device is not limited to outputting the acquired volume pulse wave as is as the measurement result, and may output only other indices obtained by calculating or measuring other specific indices based on the acquired volume pulse wave as the measurement result, or may output other obtained indices and the acquired volume pulse wave as the measurement result. Other indices include the systolic blood pressure value (maximum blood pressure), diastolic blood pressure value (minimal blood pressure value), average blood pressure value, pulse beat, AI value, and the like.

The volume pulse wave shows the cyclic fluctuation of the intravascular volume involved in the beating of the heart as wave motion, and in this regard, if the fluctuation of the intravascular volume is observed with at least a time difference herein, this can be referred to as the volume pulse wave without depending on a temporal resolution thereof. It should be recognized that a high temporal resolution is naturally required to precisely capture the volume pulse wave contained in one beat.

Generally, the blood pressure information measurement device capable of acquiring the volume pulse wave in a noninvasive manner without giving pain to the subject is classified into the following three based on the difference in measurement methods thereof.

The blood pressure information measurement device based on a first measurement method includes an ultrasonic sensor, where the fluctuation of the intra-arterial volume is captured by applying ultrasonic wave to the living body tissue including the artery and detecting the reflection wave thereof using the ultrasonic sensor, and the volume pulse wave of the artery is acquired based thereon.

The blood pressure information measurement device based on a second measurement method includes a bioelectrical impedance measurement device, where the fluctuation of the intra-arterial volume is captured by applying a very weak current to the living body tissue including the artery and measuring a bioelectrical impedance, and the volume pulse wave of the artery is acquired based thereon.

The blood pressure information measurement device based on a third measurement method includes a photoelectric sensor with a light emitting element and a light receiving element, where the fluctuation of the blood tissue amount is captured by irradiating the living body tissue including the artery with light emitted from the light emitting element, and detecting the transmitted light of the irradiated light with the light receiving element, and the volume pulse wave of the artery is acquired based thereon.

The blood pressure information measurement device based on the third measurement method using the photoelectric sensor is superior to the blood pressure information measurement devices based on the first and second measurement methods in that the measurement system can be realized with a relatively easy and convenient configuration. Furthermore, the blood pressure information measurement device based on the third measurement method can be inexpensively manufactured since the photoelectric sensor for living body used in a pulse meter, an oxygen saturation meter, and the like from the conventional art can be used for the measurement system.

The blood pressure information measurement device using such a photoelectric sensor includes one disclosed in Japanese Unexamined Patent Publication No. 6-311972 (Patent Document 1). The blood pressure information measurement device disclosed in Japanese Unexamined Patent Publication No. 6-311972 includes a pressurizing body in which a distal end is formed in a semispherical shape, a photoelectric sensor embedded at the surface of the distal end of the pressurizing body, and a pressurizing bag attached to the distal end of the pressurizing body so as to cover the photoelectric sensor. A predetermined volume of air or fluid such as liquid is sealed in advance in the pressurizing bag. In the blood pressure information measurement device, the distal end of the pressurizing body is pushed toward the measuring site in measurement, and the volume pulse wave is measured using the photoelectric sensor while maintaining a compressed state of the pressurizing bag by the pressurizing body and the measuring site.

Patent Document 1: Japanese Unexamined Patent Publication No. 6-311972

### DISCLOSURE OF THE INVENTION

### Problems to be Solved by the Invention

In a blood pressure information measurement device using a photoelectric sensor, the photoelectric sensor needs to be accurately positioned to a certain extent and then arranged with respect to a measuring site. This is because a quantity of light transmitting an artery needs to be sufficiently large in order to acquire a volume pulse wave at high accuracy using the photoelectric sensor, and to this end, positioning the arrangement position of the photoelectric sensor with respect to the artery to a certain extent is a requisite. If the photoelectric sensor is arranged shifted from the artery, the quantity of light transmitting the artery decreases and the quantity of light transmitting a living body tissue portion other than the artery increases, and thus a S/N (Signal/Noise) ratio of the obtained volume pulse wave signal degrades and an error becomes large.

Specifically, since the photoelectric sensor is configured by a pair of elements, that is, a light emitting element and a light receiving element, the light emitting element and the light receiving element are preferably positioned and arranged so that the artery is sandwiched by the light emitting element and the light receiving element when a body surface that is the measuring site is seen from a normal direction. With such an arrangement position, large quantity of light transmitting the artery can be ensured, and the S/N ratio of the obtained volume pulse wave signal can be enhanced. Such an arrangement position is realized by either a state in which the light emitting element and the light receiving element are arranged to sandwich the artery in a direction intersecting a direction the artery extends when the body surface that is the measuring site is seen from the normal direction, or a state in which the light emitting element and the light receiving element are arranged so as to overlap the artery parallel to the direction the artery extends when the body surface that is the measuring site is seen from the normal direction.

Generally, the artery is often maintained in the lightly compressed state by compressing the measuring site when measuring the volume pulse wave. This is because if the artery is lightly compressed, the detection amount of the volume pulse wave becomes large compared to when the artery is not compressed at all, and the measurement can be carried out at higher accuracy. A mechanism for lightly compressing the artery generally uses a fluid bag as disclosed in Japanese Unexamined Patent Publication No. 6-311972. As the fluid bag used to compress the measuring site includes, in addition to the fluid bag in which a predetermined volume of fluid is sealed in advance as disclosed in Japanese Unexamined Patent Publication No. 6-311972, a fluid bag which can expand and contract using a pressurization pump, an air exhaust valve, and the like can also be used.

However, in the blood pressure information measurement device including the fluid bag serving as the light compressing mechanism, the direction of the photoelectric sensor with respect to the artery may shift by the pushing state of the fluid bag with respect to the measuring site even if the positioning with respect to the artery of the photoelectric sensor is properly carried out in regard to the above positioning of the photoelectric sensor. Specifically, even if the photoelectric sensor is positioned accurately with respect to the artery in advance in measurement, the direction of the photoelectric sensor with respect to the artery may shift by body motion of the subject, the shift in the pressurizing direction, and the like, during the following measurement operation. The direction of the photoelectric sensor with respect to the artery may also shift when the fluid bag is not evenly compressed and the fluid bag is pushed in a distorted shape. For example, in the blood pressure information measurement device disclosed in Japanese Unexamined Patent Publication No. 6-311972, it is difficult to stably and continuously push the pressurizing body against the measuring site during the measurement operation of a few dozen seconds, and the direction of the photoelectric sensor has a high possibility of shifting frequently.

To prevent such shift of the direction of the photoelectric sensor, the photoelectric sensor may be arranged at the surface of the fluid bag so that the photoelectric sensor comes directly in contact with the surface of the living body. However, even with such a configuration, the shift of the direction of the photoelectric sensor cannot be completely prevented when the pushing state of the fluid bag with respect to the measuring site significantly changes or when the fluid bag is not evenly compressed and is expanded in a distorted shape. Furthermore, if the photoelectric sensor is arranged at the surface of the fluid bag, a portion where the photoelectric sensor is positioned and a portion where the photoelectric sensor is not positioned exist between the fluid bag and the measuring site, and hence the photoelectric sensor itself becomes a hindrance in compression at the portion where the photoelectric sensor is positioned, and even compression of the measuring site may not be carried out as a result. Therefore, there arises a problem that highly accurate measurement cannot be carried out even if such a configuration is adopted.

Therefore, in view of solving the above problems, it is an object of the present invention to provide a blood pressure information measurement device capable of acquiring the volume pulse wave easily and at high accuracy, and a detection unit of the same.

### Means for Solving the Problems

A detection unit for blood pressure information measurement device according to the present invention includes a compression fluid bag, a photoelectric sensor, and a fixing unit. The compression fluid bag compresses an artery in a measuring site by compressing the measuring site. The photoelectric sensor includes a light emitting portion and a light receiving portion, and radiates detection light toward the measuring site from the light emitting portion, receives the detection light transmitted through the measuring site with the light receiving portion, and outputs an output signal corresponding to a light quantity of the received detection light. The fixing unit fixes the photoelectric sensor with respect to the measuring site. The fixing unit includes a base portion with a sensor attachment surface attached with the photoelectric sensor, and a guide portion arranged projecting out from the base portion toward the sensor attachment surface side, the guide portion having a distal end directly or indirectly placed to a body surface near the measuring site with the photoelectric sensor fixed with respect to the measuring site by the fixing unit. The compression fluid bag is arranged on the sensor attachment surface so as to cover the photoelectric sensor. Furthermore, the guide portion is positioned to surround the photoelectric sensor when the fixing unit is seen from a normal direction of the sensor attachment surface.

In the detection unit for blood pressure information measurement device according to the present invention, the guide portion preferably has a wall-shape or a columnar shape.

In the detection unit for blood pressure information measurement device according to the present invention, the fixing unit preferably includes a belt member attached by being wrapped around a living body including the measuring site.

In the detection unit for blood pressure information measurement device according to the present invention, the light emitting portion and the light receiving portion are arranged in line in a longitudinal direction of the belt member.

A blood pressure information measurement device according to the present invention includes the above-described detection unit for blood pressure information device, a drive unit for causing the light emitting portion to emit light; a light receiving quantity detector for detecting a fluctuation of light receiving quantity based on an output signal outputted from the photoelectric sensor; and a volume pulse wave acquiring unit for acquiring a volume pulse wave of an artery based on information obtained by the light receiving quantity detector.

In the blood pressure information measurement device according to the present invention, the drive unit preferably causes the light emitting portion to intermittently emit pulsed light.

The blood pressure information measurement device according to the present invention further preferably includes a pressure adjustment mechanism for expanding and contracting the compression fluid bag by adjusting an internal pressure of the compression fluid bag.

The blood pressure information measurement device according to the present invention may further include an ejection wave/reflection wave acquiring unit for acquiring at least one of an ejection wave and a reflection wave of the pulse wave based on information of the volume pulse wave obtained by the volume pulse wave acquiring unit.

The blood pressure information measurement device according to the present invention may further include a compression force detector for detecting an internal pressure of the compression fluid bag, and a blood pressure value acquiring unit for acquiring a diastolic blood pressure value and a systolic blood pressure value based on information of the volume pulse wave obtained by the volume pulse wave acquiring unit and information of the pressure obtained by the compression force detector.

The blood pressure information measurement device according to the present invention may further include a compression force detector for detecting an internal pressure of the compression fluid bag, a compression force control unit for servo controlling the compression force with respect to the artery by the compression fluid bag based on information of the volume pulse wave obtained by the volume pulse wave acquiring unit, and a blood pressure value acquiring unit for acquiring a diastolic blood pressure value and a systolic blood pressure value based on information of the pressure obtained by the compression force detector.

### EFFECTS OF THE INVENTION

According to the present invention, a blood pressure information measurement device and a detection unit thereof capable of acquiring a volume pulse wave easily and at high accuracy can be realized, so that blood pressure information useful in realizing health management of a subject can be obtained at high accuracy by acquiring the volume pulse wave using the blood pressure information measurement device and the detection unit thereof.

### BRIEF DESCRIPTION OF THE DRAWINGS

FIG. 1 is a functional block diagram showing a configuration of a blood pressure information measurement device according to a first embodiment of the present invention.

FIG. 2 is a flowchart showing processing procedures of the blood pressure information measurement device according to the first embodiment of the present invention.

FIG. 3 is a schematic cross-sectional view showing an attachment state of a detection unit for blood pressure information measurement device according to the first embodiment of the present invention.

FIG. 4 is a schematic perspective view showing a configuration of a detector of the detection unit for blood pressure information measurement device shown in FIG. 2.

FIG. 5 is a schematic cross-sectional view showing a usage state of the detection unit for blood pressure information measurement device according to the first embodiment of the present invention.

FIG. 6 is a schematic perspective view showing a configuration of a detector of a detection unit for blood pressure information measurement device according to a first variant.

FIG. 7 is a schematic perspective view of a configuration of a detector of a detection unit for blood pressure information measurement device according to a second variant.

FIG. 8 is a schematic cross-sectional view showing an attachment state to a wrist of a detection unit for blood pressure information measurement device according to a third variant.

FIG. 9 is a schematic cross-sectional view showing an attachment state to the wrist of a detection unit for blood pressure information measurement device according to a fourth variant.

FIG. 10 is a functional block diagram showing a configuration of a blood pressure information measurement device according to a second embodiment of the present invention.

FIG. 11 is a flowchart showing processing procedures of the blood pressure information measurement device according to the second embodiment of the present invention.

FIG. 12 is a functional block diagram showing a configuration of a blood pressure information measurement device according to a third embodiment of the present invention.

FIG. 13 is a flowchart showing processing procedures of the blood pressure information measurement device according to the third embodiment of the present invention.

FIG. 14 is a functional block diagram showing a configuration of a blood pressure information measurement device according to a fourth embodiment of the present invention.

FIG. 15 is a flowchart showing processing procedures of the blood pressure information measurement device according to the fourth embodiment of the present invention.

### DESCRIPTION OF SYMBOLS

10A to 10C cuff
20 belt member
22a base portion
22a1 sensor attachment surface
22b guide portion
25 band-shaped tightening member
30A˜30E detector
32 fixing stand
32a base portion
32a1 sensor attachment surface
32b guide portion
40 air bladder
40a compression acting surface
50 photoelectric sensor
51 light emitting element
52 light receiving element
100A to 100D blood pressure information measurement device
110 light emitting element drive circuit
120 light receiving quantity detection circuit
131 volume pulse wave acquiring unit
132 pressure adjustment mechanism control unit
135 ejection wave/reflection wave acquiring unit
136 pressure detector
138 blood pressure value acquiring unit
140 memory
150 display unit
160 operation unit
170 power supply unit
180 air system component
181 pressurization pump
182 exhaust valve
183 pressure sensor
185 oscillation circuit
190 air tube
200 wrist
210 radius
212 radial artery
220 ulna
222 ulnar artery
230 tendon

### BEST MODE FOR CARRYING OUT THE INVENTION

Embodiments of the present invention will be hereinafter described in detail with reference to the drawings. The embodiments of the present invention described below show a case where a measuring site is a predetermined portion of a wrist, and the present invention is applied to a blood pressure information measurement device and a detection unit thereof configured to noninvasively measure a volume pulse wave of a radial artery extending in the wrist.

### First Embodiment

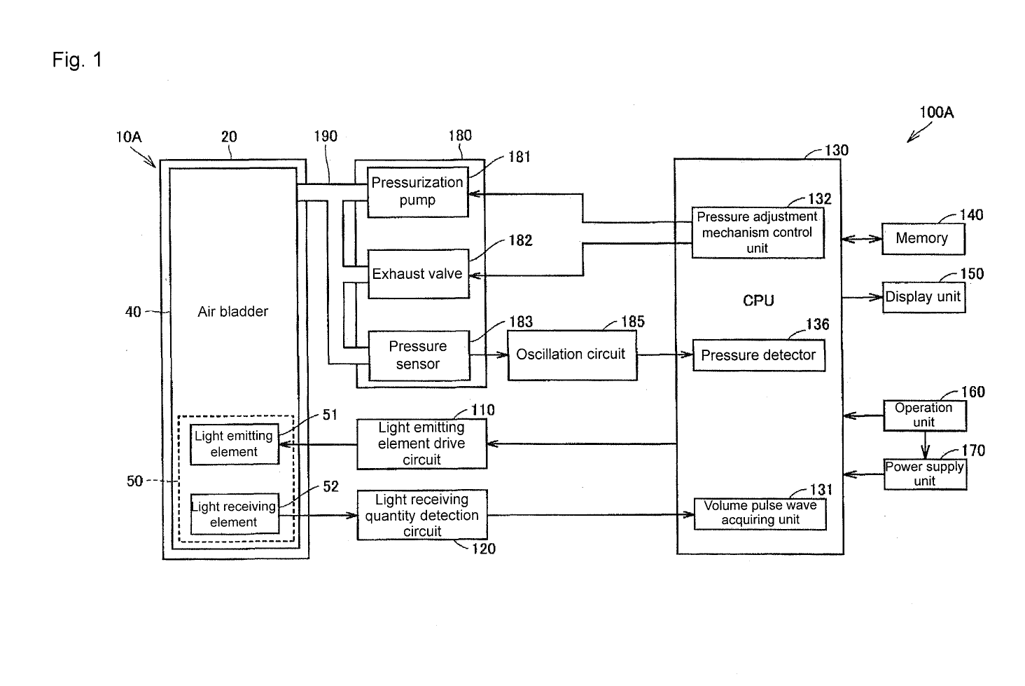

FIG. 1 is a functional block diagram showing a blood pressure information measurement device according to a first embodiment of the present invention. First, the configuration of the blood pressure information measurement device according to the present embodiment will be described with reference to FIG. 1.

As shown in FIG. 1, a blood pressure information measurement device 100A according to the present embodiment mainly includes a cuff 10A serving as a detection unit for blood pressure information measurement device, a light emitting element drive circuit 110 serving as a drive unit, a light receiving quantity detection circuit 120 serving as a light receiving quantity detector, a CPU (Central Processor Unit) 130 serving as a control unit, a memory 140, a display unit 150, an operation unit 160, a power supply unit 170, an air system component 180, an oscillation circuit 185, and an air tube 190.

The cuff 10A serving as the detection unit for blood pressure information measurement device is attached to a wrist of a subject to capture the intra-arterial volume fluctuation of the radial artery, and mainly includes a belt member 20, an air bladder 40 serving as a compression fluid bag, and a photoelectric sensor 50. The belt member 20 stably fixes the photoelectric sensor 50 to the wrist, and is made of a long band-shaped member. The air bladder 40 lightly compresses the predetermined portion of the wrist serving as the measuring site to lightly compress the radial artery, and is made of a bag-shaped member interiorly including an expanding/contracting space. The photoelectric sensor 50 includes a light emitting element 51 serving as a light emitting unit for radiating detection light toward the measuring site, and a light receiving element 52 serving as a light receiving unit for receiving the detection light transmitted through the measuring site and outputting an output signal corresponding to the light quantity of the received detection light, and optically detects a fluctuation in the blood tissue amount of the radial artery contained at the measuring site.

A semiconductor light emitting element and a semiconductor light receiving element are suitably used for the light emitting element 51 and the light receiving element 52. Near-infrared light that easily transmits through a living body tissue is preferably used for the detection light, and elements capable of emitting and receiving such near-infrared light are suitably used for the light emitting element 51 and the light receiving element 52 to accurately detect the intra-arterial volume fluctuation. More specifically, the near-infrared light around the wavelength of 940 nm is particularly suitably used for the detection light emitted from the light emitting element 51 and received by the light receiving element 52. The detection light is not limited to the near-infrared light around 940 nm, and light around the wavelength of 450 nm, light around the wavelength of 1100 nm, and the like also can be used.

The light emitting element drive circuit 110 is a circuit for causing the light emitting element 51 to emit light based on a control signal of the CPU 130, and causes the light emitting element 51 to emit light by applying a predetermined amount of current to the light emitting element 51. The current applied to the light emitting element 51 may be a direct current (DC) of about 50 mA. The light emitting element drive circuit 110 suitably uses a circuit for causing the light emitting element 51 to periodically emit pulsed light by supplying a pulse current to the light emitting element 51 at a predetermined duty. If the light emitting element 51 can thus be subjected to pulse light emission, the application power per unit time to the light emitting element 51 can be suppressed, and the temperature rise of the light emitting element 51 can be prevented. The drive frequency of the light emitting element 51 is a frequency (e.g., about 3 kHz) sufficiently higher than a frequency component (about 30 Hz) contained in the intra-arterial volume fluctuation to be detected, so that the intra-arterial volume fluctuation can be acquired more accurately.

The light receiving quantity detection circuit 120 is a circuit for generating a voltage signal corresponding to the light receiving quantity based on the signal inputted from the light receiving element 52, and outputting the same to the CPU130. The light quantity of the light detected by the light receiving element 52 changes in proportion to the intra-arterial volume, and thus the voltage signal generated by the light receiving quantity detection circuit 120 also changes in proportion to the intra-arterial volume, whereby the volume pulse wave is captured as the voltage value fluctuation. The light receiving quantity detection circuit 120 includes processing circuits such as an analog filter circuit, an amplifier circuit, and an A/D (Analog/Digital) conversion circuit, and outputs a voltage signal in which the signal inputted as an analog value is converted to a digital value.

The air system component 180 includes a pressurization pump 181, an exhaust valve 182, and a pressure sensor 183. The pressurization pump 181, the exhaust valve 182, and the pressure sensor 183 are connected to the air bladder 40 by way of the air tube 190. The pressurization pump 181 is a pressurization mechanism for expanding the air bladder 40 by sending air to the expanding/contracting space of the air bladder 40, and the exhaust valve 182 is a depressurization mechanism for contracting the air bladder 40 by exhausting air to the outside from the expanding/contracting space of the air bladder 40 in an open state. The exhaust valve 182 also functions as a pressure maintaining mechanism for maintaining the pressure of the expanding/contracting space of the air bladder 40 in a closed state. The pressurization pump 181 serving as the pressurization mechanism and the exhaust valve 182 serving as the depressurization mechanism correspond to a pressure adjustment mechanism for expanding and contracting the air bladder 40 by adjusting the internal pressure (hereinafter also referred to as cuff pressure) of the air bladder 40 serving as the compression fluid bag.

The pressure sensor 183 configures one part of a compression force detector for detecting a compression force on the wrist by detecting the internal pressure of the air bladder 40, and outputs an output signal corresponding to the internal pressure of the air bladder 40 to the oscillation circuit 185. The oscillation circuit 185 generates a signal having an oscillation frequency corresponding to the signal inputted from the pressure sensor 183, and outputs the generated signal to the CPU 130.

The CPU 130 is a site that controls the entire blood pressure information measurement device 100A. The memory 140 is configured by a ROM (Read-Only Memory) and a RAM (Random-Access Memory), and is a site for storing a program for causing the CPU 130 and the like to execute processing procedures for measuring the volume pulse wave and recording measurement results and the like. The display unit 150 is configured by an LCD (Liquid Crystal Display) and the like, and is a site for displaying the measurement results and the like. The operation unit 160 is a site for accepting an operation by the subject or the like, and inputting an external command to the CPU 130 and the power supply unit 170. The power supply unit 170 is a site for supplying power as power supply to the CPU 130.

The CPU 130 inputs the volume pulse wave information as the measurement result to the memory 140 and the display unit 150. The CPU 130 includes a pressure adjustment mechanism control unit 132 for controlling the pressure adjustment mechanism, where the above-described operation of the pressurization pump 181 and the exhaust valve 182 is controlled based on the control signal from the pressure adjustment mechanism control unit 132. The CPU 130 includes a pressure detector 136 for detecting the internal pressure of the air bladder 40, which pressure detector 136 detects the internal pressure of the air bladder 40 based on the signal inputted from the oscillation circuit 185 to thereby measure the compression force on the artery by the air bladder 40. The CPU 130 inputs the control signal for driving the light emitting element 51 to the light emitting element drive circuit 120. Furthermore, the CPU 130 include a volume pulse wave acquiring unit 131 for acquiring the volume pulse wave, wherein the volume pulse wave acquiring unit 131 acquires the volume pulse wave based on the voltage signal inputted from the light receiving quantity detection circuit 120. The volume pulse wave information acquired by the volume pulse wave acquiring unit 131 is inputted to the memory 140 and the display unit 150 as the measurement result.

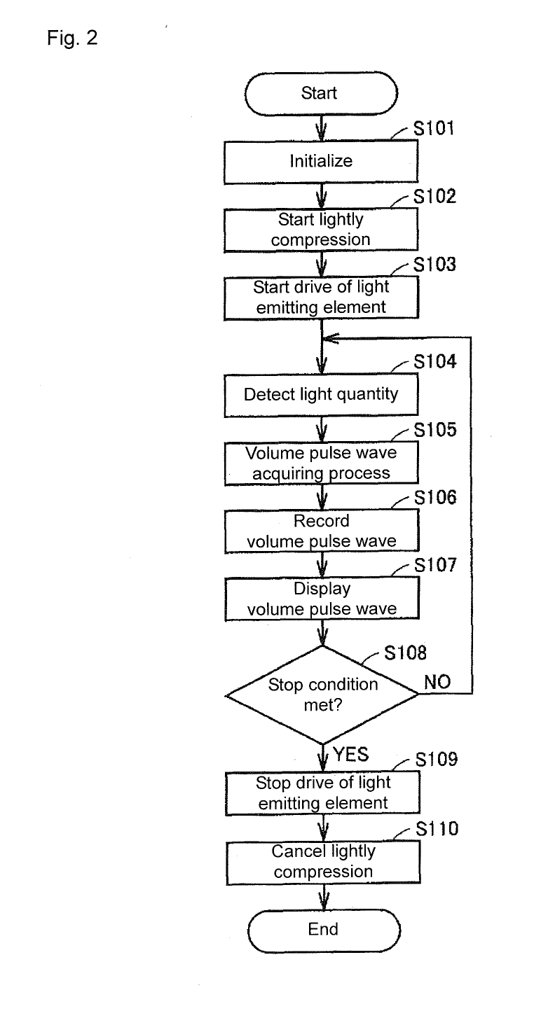

FIG. 2 is a flowchart showing the processing procedures of the blood pressure information measurement device according to the present embodiment. The processing procedures of the blood pressure information measurement device according to the present embodiment will be described below with reference to FIG. 2. The program according to the flowchart is stored in advance in the memory 140 shown in FIG. 1, and the process is implemented when the CPU 130 reads out the program from the memory 140 and executes the same.

As shown in FIG. 2, when the subject operates the operation unit 160 of the blood pressure information measurement device 100A and inputs a command to turn ON power, power as power supply is supplied from the power supply unit 170 to the CPU 130, so that the CPU 130 is driven and initialization of the blood pressure information measurement device 100A is performed (step S101). The subject attaches the cuff 10A serving as the above-described detection unit for blood pressure information measurement device to the wrist in advance.

When the subject operates an operation button of the operation unit 160 of the blood pressure information measurement device 100A and inputs a command to start measurement, the CPU 130 controls the pressurization pump 181 and the exhaust valve 182 through the pressure adjustment mechanism control unit 132 to start pressurizing the air bladder 40. The air is thereby sent to the air bladder 40, and the air bladder 40 starts to lightly compress the measuring site (step S102). The air bladder 40 is pressurized using the pressurization pump 181 until the air bladder 40 reaches a predetermined internal pressure. More specifically, the air bladder 40 is pressurized up to an extent of lightly compressing the radial artery 212, and thereafter the internal pressure of the air bladder 40 is maintained and the lightly compressing state is held.

The CPU 130 then starts to drive the light emitting element 51 through the light emitting element drive circuit 110 (step S103). The detection light is then radiated from the light emitting element 51 toward the measuring site including the radial artery 212. In parallel with the drive of the light emitting element 51, the light receiving quantity detection circuit 120 generates a voltage signal digitized based on the signal inputted from the light receiving element 52 (step S104), and inputs the same to the CPU 130. The CPU 130 acquires the volume pulse wave by the volume pulse wave acquiring unit 131 based on the inputted voltage signal (step S105). The acquired volume pulse wave is stored in the memory 140 as the measurement result (step S106), and thereafter, displayed on the display unit 150 (step S107). The display unit 150 displays the volume pulse wave as a waveform.

The series of operations including step S104 to step S107 are repeatedly performed until a predetermined stop condition (e.g., input of a command to stop measurement by the subject, elapse of a set time by a timer circuit, and the like) is met (NO in step S108). When the predetermined stop condition is met (YES in step S108), the CPU 130 makes an instruction to cancel the drive of the light emitting element 51 to the light emitting element drive circuit 110, and outputs an instruction to have the exhaust valve 182 in the open state. The drive of the light emitting element 51 is then stopped (step S109), and the air in the air bladder 40 is exhausted to release the lightly compressed state (step S110). Then, the blood pressure information measurement device 100A is in a standby state, and stops the supply of power as the power supply after waiting for the input of command to turn OFF the power by the operation unit 160 of the subject. Accordingly, the volume pulse wave that changes momentarily can be acquired in real time.

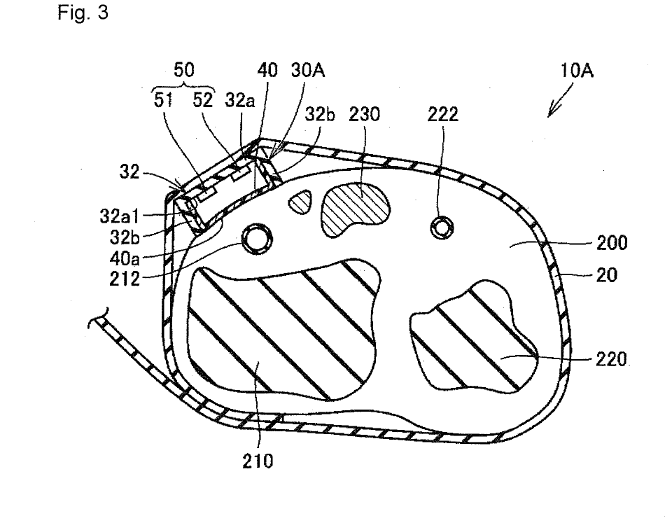

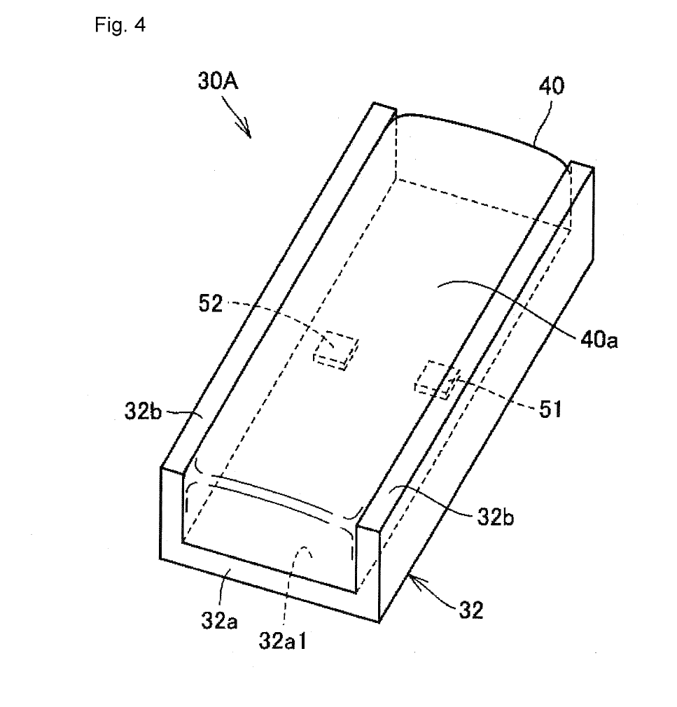

FIG. 3 is a schematic cross-sectional view showing a state in which the detection unit for blood pressure information measurement device according to the present embodiment is attached to the wrist. FIG. 4 is a schematic perspective view showing a configuration of a detector of the detection unit for blood pressure information measurement device shown in FIG. 3. With reference to FIGS. 3 and 4, the specific structure of the detection unit for blood pressure information measurement device according to the present embodiment, and a state in which the detection unit for blood pressure information measurement device is attached to the wrist will be described. FIG. 3 is a cross-sectional view when a wrist of a left hand is seen from a central side toward a peripheral side, and FIG. 4 is a schematic perspective view when the detector shown in FIG. 3 is seen from the living body side.

As shown in FIG. 3, a radius 210, the radial artery 212, an ulna 220, an ulnar artery 222, and a tendon 230 are positioned in the wrist 200 as characteristic living body tissues. The cuff 10A serving as the detection unit for blood pressure information measurement device according to the present embodiment is attached in a state wrapped around the wrist 200.

The cuff 10A is formed in a shape suited for the attachment to the wrist 200 of the subject, and includes the belt member 20, and a detector 30A attached to a predetermined position of the belt member 20. The belt member 20 is made of a band-shaped member having a length capable of being wrapped around the wrist 200, and is attached in a state wrapped around the wrist 200 by engaging a portion closer to one end in a longitudinal direction and a surface fastener (not shown) arranged at a portion closer to the other end.

As shown in FIGS. 3 and 4, the detector 30A mainly includes a fixing stand 32, the air bladder 40, and the photoelectric sensor 50 including the light emitting element 51 and the light receiving element 52. The fixing stand 32 includes a base portion 32a to be attached to the belt member 20, and a guide portion 32b arranged uprising from the end of the base portion 32a. The base portion 32a has a plate-shape with a rectangular shape in plan view, and is attached to the belt member such that the longitudinal direction thereof intersects (substantially orthogonally) with a length direction of the belt member 20. One main surface of the base portion 32a is a sensor attachment surface 32a1 where the photoelectric sensor 50 is attached, and the guide portion 32b is arranged projecting toward the sensor attachment surface 32a1 (toward the measuring site side in the attached state) from the base portion 32a. The guide portion 32b is arranged uprising from a pair of opposing sides of the base portion 32a, and has a wall shape. The fixing stand 32 is fixed to the belt member 20 by adhesion, welding, screw-fitting, and the like.

The photoelectric sensor 50 including the light emitting element 51 and the light receiving element 52 is arranged substantially at the central part in the longitudinal direction of the sensor attachment surface 32a1 of the fixing stand 32. More specifically, the light emitting element 51 and the light receiving element 52 are arranged in line with a predetermined distance in the short-side direction of the base portion 32a (i.e., length direction of the belt member 20). A distance between the light emitting element 51 and the light receiving element 52, which are arranged spaced apart, is preferably about two or more times a distance from the sensor attachment surface 32a1 to the measuring site (i.e., height of guide portion 32b), for example, to enable stable irradiation of the detection light to the radial artery 212 positioned under the skin, and to reliably receive the detection light transmitted or reflected by the measuring site. If such a distance is short, the detection light emitted from the light emitting element 51 is substantially totally reflected at the surface of the skin, and thus the detection light may not reach the radial artery 212 and accurate measurement may not be made.

The air bladder 40 is arranged on the sensor attachment surface 32a1 of the base portion 32a attached with the photoelectric sensor 50. The air bladder 40 is arranged to fill the space configured by the base portion 32a and the guide portion 32b of the fixing stand 32, and the photoelectric sensor 50 is in a state completely covered by the air bladder 40. The air bladder 40 is made of a material capable of transmitting the detection light emitted from the light emitting element 51, and hence most of the detection light emitted from the light emitting element 51 is transmitted through the air bladder 40 and radiated on the measuring site. The air bladder 40 is connected to the air tube 190 by a connection member such as a nipple (not shown), and connected to the air system component 180 through the air tube 190. The air bladder 40 may be in a state a certain amount of air is sealed in advance, or may be in a state the air is completely exhausted.

The guide portion 32b having a shape of a pair of wall shape is arranged to surround the photoelectric sensor 50 when the sensor attachment surface 32a1 is seen from a normal direction. In the detection unit for blood pressure information measurement device according to the present embodiment, the air bladder 40 is also surrounded by the guide portion 32b having a shape of a pair of wall shapes. The main surface positioned substantially parallel with the sensor attachment surface 32a1 of the exposing surface of the air bladder 40 functions as a compression acting surface 40a for lightly compressing the radial artery 212 by lightly compressing a predetermined portion of the wrist 200 serving as the measuring site.

As shown in FIG. 3, upon the attachment of the cuff 10A, the detector 30A is positioned and arranged in a state where a lower surface thereof (more specifically, the compression acting surface 40a of the air bladder 40) is in contact with the skin immediately above the portion where the radial artery 212 is positioned. Such positioning is made by adjusting the attachment position of the belt member 20 with respect to the peripheral direction of the wrist 200. At this time, the center position in the short direction of the detector 30A is arranged immediately above the radial artery 212 traveling in the wrist 200. If the detector 30A is positioned and arranged in such a manner, the light emitting element 51 and the light receiving element 52 are arranged so as to sandwich the radial artery 212 in the direction intersecting the direction the radial artery 212 extends when the body surface is seen from the normal direction. The portion where the radial artery 212 is positioned is specified by hand examination, and the like.

After the positioning, the belt member 20 is fixed using the surface fastener (not shown) to realize the attachment state shown in FIG. 3. In such an attachment state, the detector 30A is fixed while being pushed toward the wrist 200. The distal end of the guide portion 32b of the fixing stand 32 is placed on the body surface near the measuring site in the attachment state, so that a relative positional relationship between the photoelectric sensor 50 (more specifically, light emitting element 51 and light receiving element 52) arranged in the detector 30A and the radial artery 212 can be held. Therefore, the belt member 20 and the fixing stand 32 function as a fixing unit for fixing the photoelectric sensor 50 with respect to the measuring site.

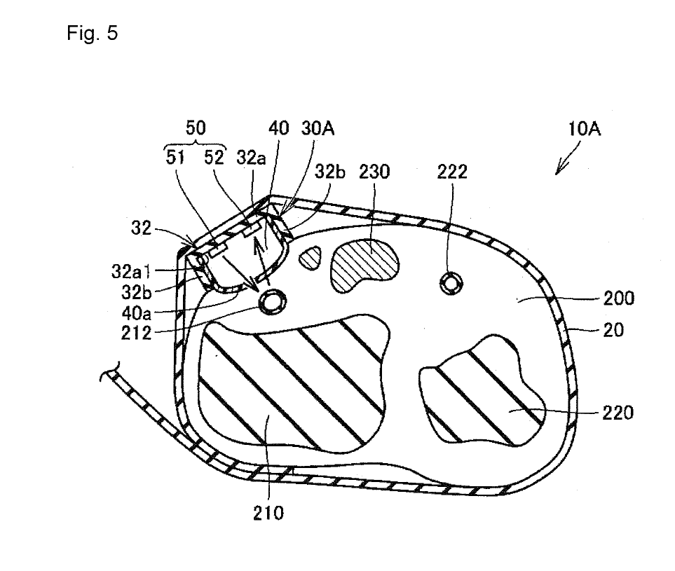

FIG. 5 is a schematic cross-sectional view showing a usage state of the detection unit for blood pressure information measurement device according to the present embodiment. With reference to FIG. 5, the operation of the detection unit for blood pressure information measurement device when the volume pulse wave is measured and the state of the wrist in the usage state will be described.

As shown in FIG. 5, when the air bladder 40 is pressurized to a predetermined pressure, the compression acting surface 40a of the air bladder 40 bulges out from the fixing stand 32 so that the measuring site is lightly compressed, and the radial artery 212 is also lightly compressed therewith. Since the detector 30A is held in a state pushed toward the measuring site by the belt member 20, the distal end of the guide portion 32b of the fixing stand 32 is maintained in a state in contact with the skin near the measuring site even in the compressed state, and the relative positional relationship between the photoelectric sensor 50 and the radial artery 212 is also maintained.

In this state, as shown with the arrow in FIG. 5, the detection light is radiated from the light emitting element 51 toward the radial artery 212 in the measuring site, and the detection light transmitted through the radial artery 212 is received by the light receiving element 52. Accordingly, the intra-arterial volume fluctuation is optically captured, and the volume pulse wave can be measured.

In the blood pressure information measurement device 100A and the cuff 10A serving as the detection unit thereof according to the present embodiment described above, the guide portion 32b is arranged uprising from the base portion 32a of the fixing stand 32 attached with the photoelectric sensor 50, and the distal end of the guide portion 32b is placed on the body surface near the measuring site in the attachment state of the cuff 10A to the wrist 200. Therefore, the relative positional relationship of the photoelectric sensor 50 and the radial artery 212 is always held during the measurement operation, and hence the problem of the conventional art in that the direction of the photoelectric sensor shifts with respect to the radial artery does not arise, and the volume pulse wave can be acquired at high accuracy. Since the device configuration is not complicating, the blood pressure information measurement device and the detection unit thereof capable of performing highly accurate measurement easily and conveniently can be obtained.

In the blood pressure information measurement device 100A and the cuff 10A serving as the detection unit thereof according to the present embodiment described above, the fixing stand 32 configuring the detector 30A is pushed toward the wrist 200 using the belt member 20. Therefore, the attachment state is stably maintained even during the measurement operation of a few dozen seconds, and the volume pulse wave of high accuracy can be acquired from such a standpoint. Furthermore, since the light emitting element 51 and the light receiving element 52 are arranged in line in the longitudinal direction of the belt member 20, the radial artery 212 can be more easily arranged between the light emitting element 51 and the light receiving element 52 in the attachment state, and the detector 30A can be positioned more easily with respect to the measuring site.

In the blood pressure information measurement device 100A and the cuff 10A serving as the detection unit thereof according to the present embodiment described above, the guide portion 32b has a wall-shape and the photoelectric sensor 50 is surrounded by the guide portion 32b, and thus the attachment state can be more stably maintained.

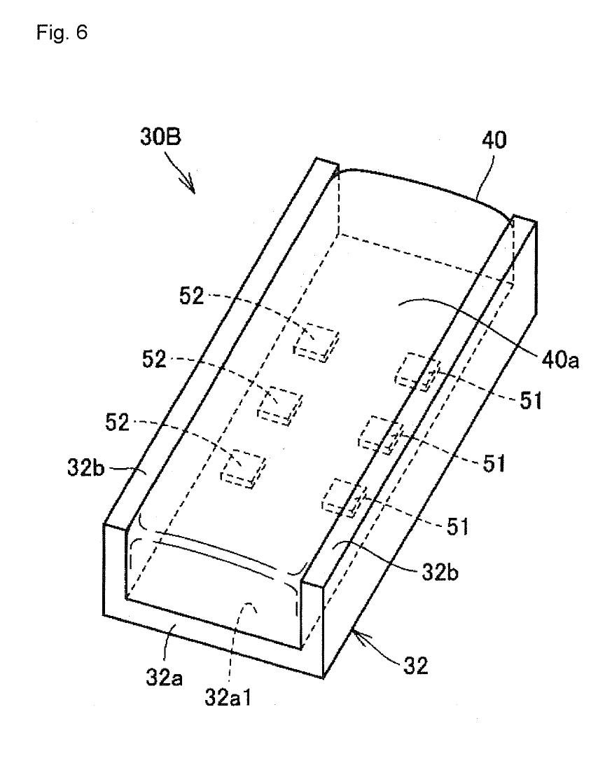

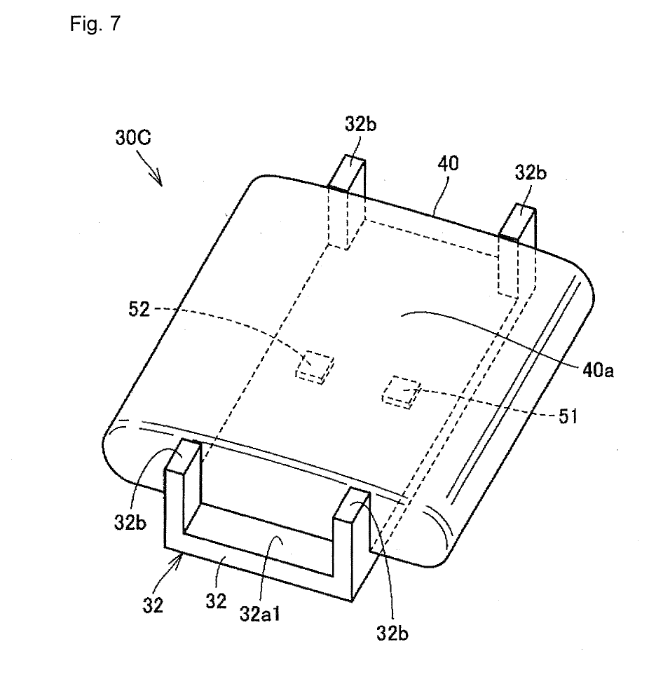

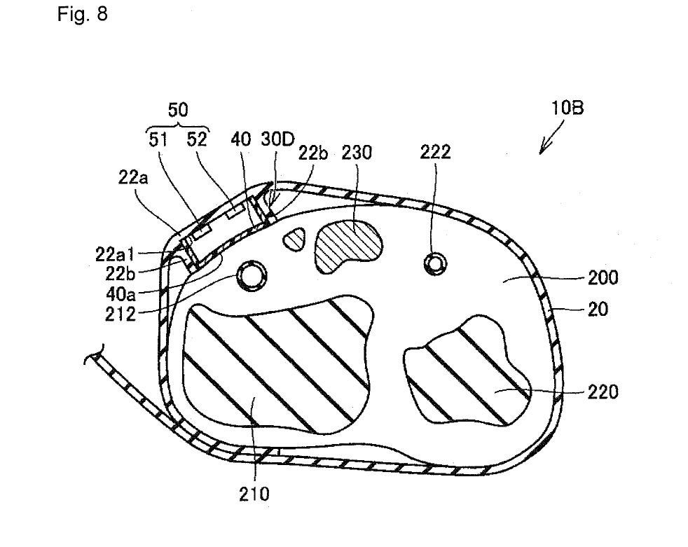

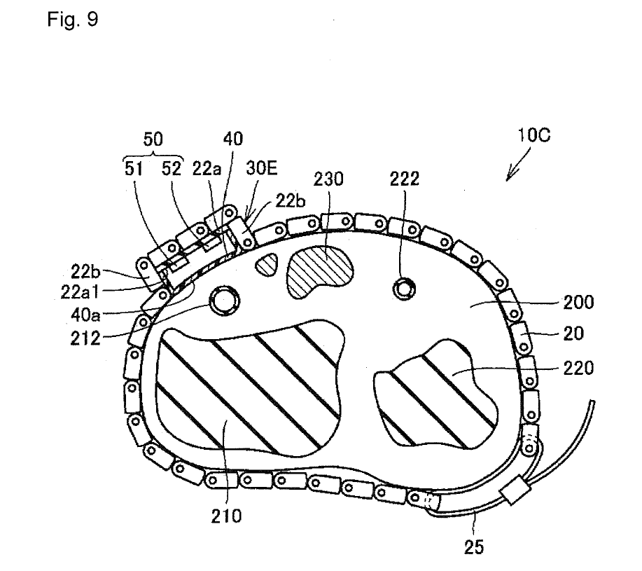

Variants of the detection unit for blood pressure information measurement device according to the present embodiment described above will now be described with reference to FIGS. 6 to 9. FIG. 6 is a schematic perspective view showing a configuration of a detector of a detection unit for blood pressure information measurement device according to a first variant, and FIG. 7 is a schematic perspective view of a configuration of a detector of a detection unit for blood pressure information measurement device according to a second variant. FIG. 8 is a schematic cross-sectional view showing an attachment state to the wrist of a detection unit for blood pressure information measurement device according to a third variant, and FIG. 9 is a schematic cross-sectional view showing an attachment state to the wrist of a detection unit for blood pressure information measurement device according to a fourth variant. The same reference numerals are denoted in the figures for portions similar to the detection unit for blood pressure information measurement device according to the present embodiment described above, and the description thereof will not be repeated.

As shown in FIG. 6, the detection unit for blood pressure information measurement device according to the first variant includes the light emitting element 51 serving as a light emitting unit and a light receiving element 52 serving as a light receiving unit in plurals in the detector 30B. The plurality of light emitting elements 51 are arranged in line along the longitudinal direction of the sensor attachment surface 32a1, and the plurality of light receiving elements 52 are arranged in line along the longitudinal direction of the sensor attachment surface 32a1 in correspondence with the plurality of light emitting elements 51. Thus, if the plurality of light emitting elements 51 and the light receiving elements 52 are arranged in the detector 30B, the volume pulse wave can be acquired at higher accuracy, and a degree of freedom in positioning can be enhanced, so that the blood pressure information measurement device and the detection unit thereof capable of acquiring the volume pulse wave at high accuracy more easily and conveniently can be obtained.

In FIG. 6, a case where three light emitting elements 51 and three light receiving elements 52 are arranged in the detector 30B is shown by way of example, but the number of light emitting elements and light receiving elements to be arranged in the detector is not particularly limited, and the same number of light emitting elements and light receiving elements does not need to be arranged. The light emitting element and the light receiving element do not need to be arranged lined to each other, and may be alternately arranged. Furthermore, the light emitting element and the light receiving element may be arranged in line in the longitudinal direction of the sensor attachment surface or may be arranged in the diagonal direction. Thus, the number, layout, and the like of the light emitting element and the light receiving element may be appropriately changed.

As shown in FIG. 7, in the detection unit for blood pressure information measurement device according to the second variant, the fixing stand 32 of the detector 30C is configured by the base portion 32a and the guide portion 32b of a columnar shape, where the guide portion 32b having a columnar shape is arranged uprising toward the sensor attachment surface 32a1 side from the four corners of the base portion 32a having a plate shape. Furthermore, the air bladder 40 has one part thereof projecting to both sides in the short direction from the sensor attachment surface 32a1 of the fixing stand 32. With such a configuration, a wider range of the wrist can be compressed with the air bladder 40 and a more stable volume pulse wave can be measured in addition to the effects described in the present embodiment above.

As shown in FIG. 8, in the cuff 10B serving as the detection unit for blood pressure information measurement device according to the third variant, the fixing stand and the belt member are configured by a single integrated member, so that the detector 30D is configured as one part of the belt member 20. More specifically, with the predetermined position in the longitudinal direction of the belt member 20 as the base portion 22a, the photoelectric sensor 50 is attached to the main surface on the inner peripheral surface side to become the sensor attachment surface 22a1, and the guide portion 22b is formed toward the inner side at the belt member 20 to surround the sensor attachment surface 22a1, thereby providing the belt member 20 a role of the fixing stand. The air bladder 40 is arranged to fill the space formed by the base portion 22a and the guide portion 22b, which are part of the belt member 20, and to cover the photoelectric sensor 50. With such a configuration, the number of components can be reduced and the blood pressure information measurement device and the detection unit can be configured with a simple configuration in addition to the effects described in the present embodiment above.

As shown in FIG. 9, in the cuff 10C serving as the detection unit for blood pressure information measurement device according to the fourth variant, a plurality of fixing tool parts is coupled to each other with a coupling pin to be used as the belt member 20. Specifically, the belt member 20 is configured to fit the wrist 200 in the attachment state by connecting the adjacent fixing tool parts with the coupling pin to become the belt member 20 that can be changed to an arbitrary shape. The fixing tool part of a portion corresponding to a detector 30E realizes the shape shown in FIG. 9 by fixing with a shape fixing coupling pin (not shown) for having the shape of the relevant portion to a predetermined shape in addition to the coupling pin. More specifically, the fixing tool part at one part of the belt member 20 is fixed to a convex shape with the shape fixing coupling pin (not shown), the guide portion 22b is formed by the side wall of the convex portion, and the portion attached with the base portion 22a is formed by the bottom of the convex portion. With such a configuration as well, effects similar to the effects described in the present embodiment above can be obtained. If a plurality of fixing tool parts coupled to each other with a coupling pin are used as the belt member 20, a band-shaped tightening member 25 shown in the figure is preferably used in place of the surface fastener used in the above present embodiment for the fixing unit for fixing the belt member 20.

### Second Embodiment

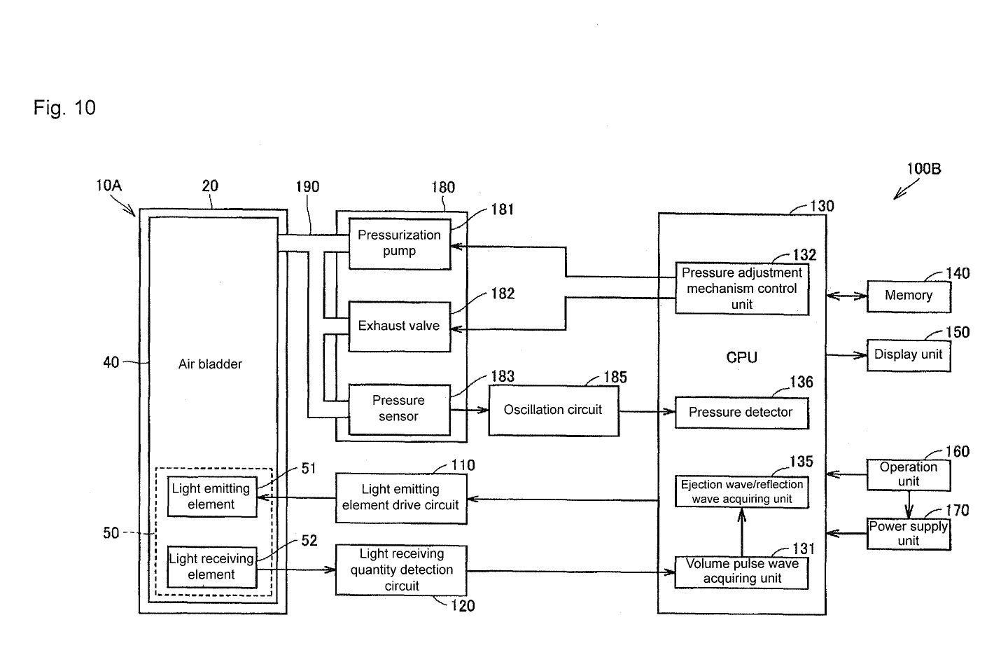

FIG. 10 is a functional block diagram showing a configuration of a blood pressure information measurement device according to a second embodiment of the present invention. A configuration of a blood pressure information measurement device 1008 according to the present embodiment will be described with reference to FIG. 10. The same reference numerals are denoted for the portions similar to the blood pressure information measurement device 100A in the first embodiment, and the description thereof will not be repeated herein.

As shown in FIG. 10, in the blood pressure information measurement device 100B according to the present embodiment, an ejection wave/reflection wave acquiring unit 135 is arranged in the CPU 130. The ejection wave/reflection wave acquiring unit 135 analyzes based on the information of the volume pulse wave obtained by the volume pulse wave acquiring unit 131 and calculates at least one of the ejection wave and the reflection wave of the radial artery.

The ejection wave is a pulse wave component generated when the heart contracts, and the reflection wave is a pulse wave component generated when the ejection wave is reflected at each area of the artery. An AI value derived from the ejection wave and the reflection wave is known as an index correlated with the extensibility of the artery and the degree of the heart load.

Measuring the volume pulse wave obtained by the volume pulse wave acquiring unit 131 at high accuracy is essential in calculating the ejection wave or the reflection wave at high accuracy. Thus, similar to the blood pressure information measurement device 100A according to the first embodiment, the blood pressure information measurement device 100B according to the present embodiment includes the air bladder 40 serving as a compression fluid bag, and the air system component 180 serving as a pressure adjustment mechanism, which are configured to enable the measurement of the volume pulse wave at an optimum amplitude.

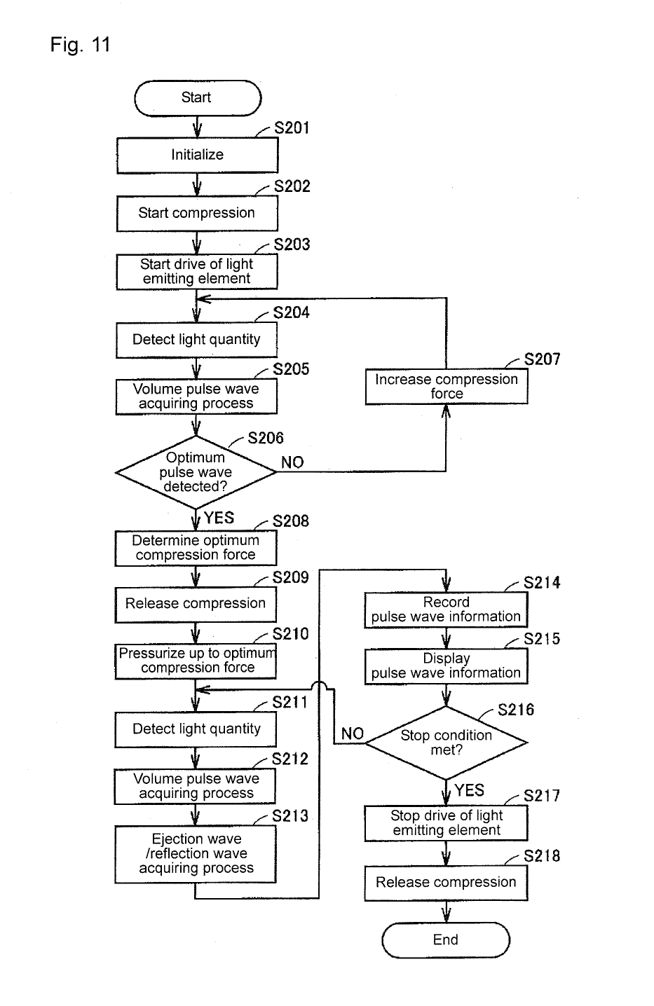

FIG. 11 is a flowchart showing the processing procedures of the blood pressure information measurement device according to the present embodiment. The processing procedures of the blood pressure information measurement device 100B according to the present embodiment will be described below with reference to FIG. 11. The program according to the flowchart is stored in advance in the memory 140 shown in FIG. 10, and the process is implemented when the CPU 130 reads out the program from the memory 140 and executes the same.

As shown in FIG. 11, when the subject operates the operation unit 160 of the blood pressure information measurement device 100B and inputs a command to turn ON power, power as power supply is supplied from the power supply unit 170 to the CPU 130, so that the CPU 130 is driven and initialization of the blood pressure information measurement device 100B is performed (step S201). The subject attaches the cuff 10A serving as the detection unit for blood pressure information measurement device to the wrist in advance.

When the subject operates the operation button of the operation unit 160 of the blood pressure information measurement device 100B and inputs a command to start measurement, the CPU 130 controls the pressurization pump 181 and the exhaust valve 182 through the pressure adjustment mechanism control unit 132 to start pressurizing the air bladder 40. The air is thereby sent to the air bladder 40, and the air bladder 40 starts to lightly compress the measuring site (step S202). The air bladder 40 is pressurized using the pressurization pump 181 until the air bladder 40 reaches a predetermined internal pressure. More specifically, the air bladder 40 is pressurized up to an extent of lightly compressing the radial artery, and thereafter the internal pressure of the air bladder 40 is maintained and the lightly compressing state is held.

The CPU 130 then starts to drive the light emitting element 51 through the light emitting element drive circuit 110 (step S203). The detection light is then radiated from the light emitting element 51 toward the measuring site including the radial artery. In parallel with the drive of the light emitting element 51, the light receiving quantity detection circuit 120 generates a voltage signal digitized based on the signal inputted from the light receiving element 52 (step S204), and inputs the same to the CPU 130. The CPU 130 acquires the volume pulse wave by the volume pulse wave acquiring unit 131 based on the inputted voltage signal (step S205).

The CPU 130 determines in step S206 whether the amplitude of the measured volume pulse wave has a magnitude suited for the calculation of the ejection wave/reflection wave, and when determined that the magnitude of the amplitude is insufficient (NO in step S206), the CPU 130 proceeds to step S207 to increase the compression force on the radial artery by a predetermined level (i.e., increase the internal pressure of the air bladder 40 by a predetermined level), and returns to step S204. When determined that the magnitude of the amplitude is sufficient (YES in step S206), the CPU 130 proceeds to step S208 and determines the relevant cuff pressure as a cuff pressure at which the optimum compression force can be obtained.

The CPU 130 then outputs a command for rapid exhaust with respect to the air system component 180, once releases the compression of the radial artery by the air bladder 40 (step S209), and again drives the air system component 180 to raise the internal pressure of the air bladder 40 to the pressure at which the optimum compressing force determined in step S208 is obtained (step S210). Thereafter, the CPU 130 acquires the volume pulse wave by the volume pulse wave acquiring unit 131 based on the voltage signal inputted from the light receiving quantity detection circuit 120 (steps S211, S212). The acquired volume pulse wave is then inputted to the ejection wave/reflection wave acquiring unit 135, and the calculation of the ejection wave or/and reflection wave is carried out in the ejection wave/reflection wave acquiring unit 135 (step S213). The blood pressure information including the acquired volume pulse wave and the calculated ejection wave or/and reflection wave are stored in the memory 140 as the measurement result (step S214), and thereafter, displayed on the display unit 150 (step S215). The display unit 150 displays the volume pulse wave or the ejection wave or/and reflection wave as a numerical value or a waveform.

The series of operations including step S211 to step S215 are repeatedly performed until a predetermined stop condition (e.g., input of a command to stop measurement by the subject, elapse of a set time by a timer circuit, and the like) is met (NO in step S216). When the predetermined stop condition is met (YES in step S216), the CPU 130 makes an instruction to cancel the drive of the light emitting element 51 to the light emitting element drive circuit 110, and outputs an instruction to have the exhaust valve 182 in the open state. Accordingly, the drive of the light emitting element 51 is stopped (step S217), and the air in the air bladder 40 is exhausted to release the lightly compressed state (step S218). Then, the blood pressure information measurement device 100B is in a standby state, and stops the supply of power as the power supply after waiting for the input of command to turn OFF the power by the operation unit 160 of the subject. Accordingly, the volume pulse wave that changes momentarily as well as the ejection wave or/and reflection wave can be measured in real time.

The blood pressure information measurement device 100B described above may be a blood pressure information measurement device capable of measuring the ejection wave and the reflection wave. In the blood pressure information measurement device 100B according to the present embodiment as well, the relative positional relationship between the photoelectric sensor 50 and the radial artery is constantly held during the measurement operation with the detection unit for blood pressure information measurement device having a configuration similar to the cuff 10F described in the first embodiment, whereby the shift in the direction of the photoelectric sensor with respect to the radial artery, which has been a conventional problem, does not arise and a blood pressure information measurement device capable of acquiring the ejection wave and the reflection wave easily and at high accuracy can be realized.

### Third Embodiment

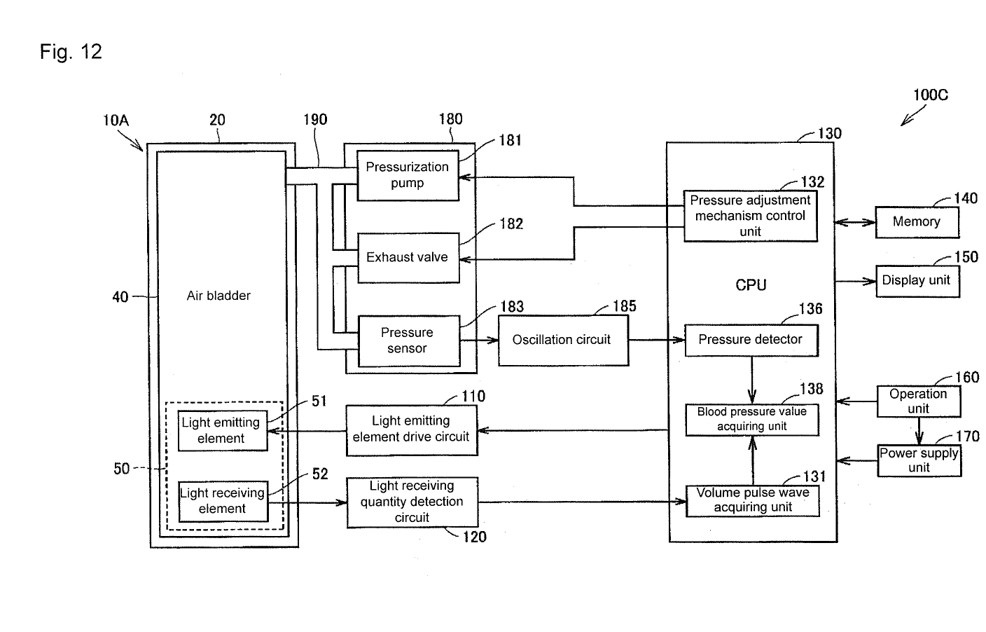

FIG. 12 is a functional block diagram showing a configuration of a blood pressure information measurement device according to a third embodiment of the present invention. A configuration of a blood pressure information measurement device 100C according to the present embodiment will be described with reference to FIG. 12. The same reference numerals are denoted for the portions similar to the blood pressure information measurement device 100A in the first embodiment, and the description thereof will not be repeated herein.

The blood pressure information measurement device 100C according to the present embodiment is a blood pressure information measurement device including a blood pressure value acquiring function of volume vibration method. As shown in FIG. 12, in the blood pressure information measurement device 100C according to the present embodiment, a blood pressure value acquiring unit 138 is arranged in the CPU 130. The blood pressure value acquiring unit 138 acquires the systolic blood pressure value and the diastolic blood pressure value based on the information of the volume pulse wave obtained by the volume pulse wave acquiring unit 131 and the pressure information obtained by the pressure detector 136.

The systolic blood pressure value and the diastolic blood pressure value are blood pressure values correlated with a point where the pulsation of the artery significantly changes in the process of fluctuating the compression force by the cuff, and are blood pressure values measured by applying a predetermined algorithm based thereon, which are known as representative indices of health management from the conventional art.

The blood pressure information measurement device 100C according to the present embodiment includes the air system component 180 having a configuration similar to the blood pressure information measurement device 100A according to the first embodiment, and uses the air system component 180 to fluctuate the compression force by the air bladder 40 on the radial artery and acquires the volume pulse wave while detecting the compression force as an internal pressure of the air bladder 40 (cuff pressure), so that the systolic blood pressure value and the diastolic blood pressure value can be acquired by the blood pressure value acquiring unit 138 based thereon.

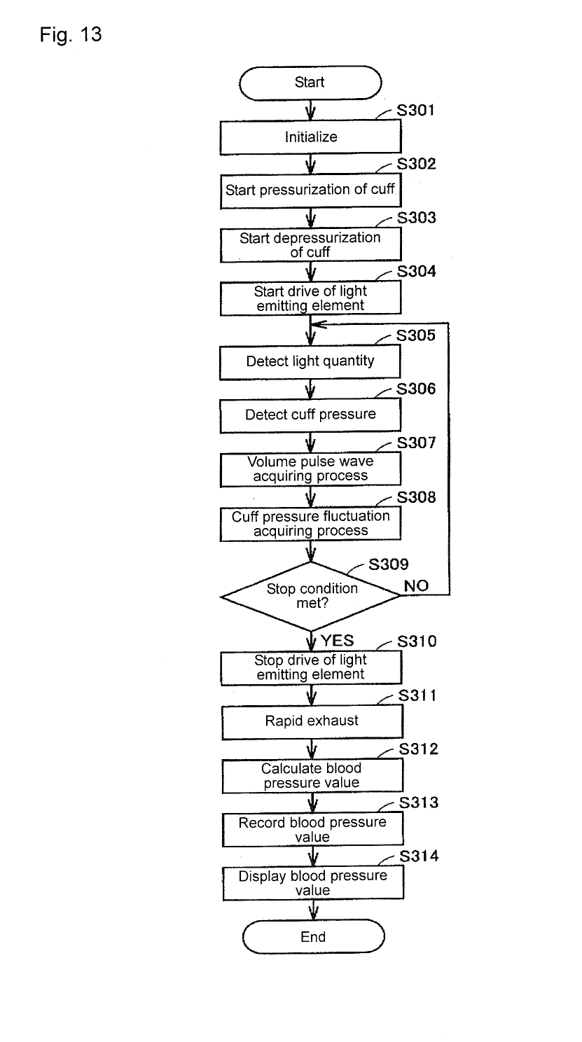

FIG. 13 is a flowchart showing the processing procedures of the blood pressure information measurement device according to the present embodiment. The processing procedures of the blood pressure information measurement device 100C according to the present embodiment will be described below with reference to FIG. 13. The program according to the flowchart is stored in advance in the memory 140 shown in FIG. 12, and the process is implemented when the CPU 130 reads out the program from the memory 140 and executes the same.

As shown in FIG. 13, when the subject operates the operation unit 160 of the blood pressure information measurement device 100C and inputs a command to turn ON power, power as power supply is supplied from the power supply unit 170 to the CPU 130, so that the CPU 130 is driven and initialization of the blood pressure information measurement device 100C is performed (step S301). The subject attaches the cuff 10A serving as the detection unit for blood pressure information measurement device to the wrist in advance.

When the subject operates the operation button of the operation unit 160 of the blood pressure information measurement device 100C and inputs a command to start measurement, the pressurization pump 181 is driven by the pressure adjustment mechanism control unit 132 arranged in the CPU 130 so that air is sent to the air bladder 40 thereby gradually raising the cuff pressure (step S302). The cuff pressure is detected by the pressure sensor 183, and when detected that the cuff pressure reached a predetermined level, the CPU 130 stops the pressurization pump 181, and gradually opens the closed exhaust valve 182 to gradually exhaust the air in the air bladder 40, thereby gradually depressurizing the cuff pressure (step S303).

In the low-speed depressurization process of the cuff pressure, the CPU 130 starts to drive the light emitting element 51 through the light emitting element drive circuit 110 (step S304), so that the detection light is radiated from the light emitting element 51 toward the measuring site including the radial artery. In parallel with the drive of the light emitting element 51, the light receiving quantity detection circuit 120 generates a voltage signal digitized based on the signal inputted from the light receiving element 52 (step S305), and inputs the same to the CPU 130. The CPU 130 detects the pressure information outputted from the pressure sensor 183 to the oscillation circuit 185 (step S306). The volume pulse wave is thereby acquired by the volume pulse wave acquiring unit 131 and the cuff pressure is acquired by the pressure detector (steps S307, S308).

The series of operations including step S305 to step S308 are repeatedly performed until a predetermined stop condition (e.g., elapse of a set time by a timer circuit, whether the cuff pressure is depressurized to a predetermined level, and the like) is met (NO in step S309). When the predetermined stop condition is met (YES in step S309), the CPU 130 makes an instruction to cancel the drive of the light emitting element 51 to the light emitting element drive circuit 110 (S310).

The CPU 130 then outputs a command for rapid exhaust with respect to the air system component 180, releases the compression of the radial artery by the air bladder 40 (step S311), inputs the volume pulse wave obtained in step S307 to the blood pressure value acquiring unit 138 and also inputs the cuff pressure obtained in step S308 to the blood pressure value acquiring unit 138 to acquire the systolic blood pressure value and the diastolic blood pressure value (step S312). The blood pressure value acquiring unit 138 applies a predetermined algorithm to the volume pulse wave acquired in the process of fluctuating the compression force by the cuff to acquire the systolic blood pressure value and the diastolic blood pressure value. The systolic blood pressure value and the diastolic blood pressure value acquired by the blood pressure value acquiring unit 138 are then stored in the memory 140 as the measurement result (step S313), and thereafter, the measurement results are displayed on the display unit 150 (step S314). The display unit 150 displays the systolic blood pressure value and the diastolic blood pressure value, for example, as a numerical value. After recording and display of the blood pressure information, the blood pressure information measurement device 100C is in a standby state and stops the supply of power as the power supply after waiting for the input of command to turn OFF the power by the operation unit 160 of the subject.

The blood pressure information measurement device 100C described above may be a blood pressure information measurement device capable of measuring the systolic blood pressure value and the diastolic blood pressure value. In the blood pressure information measurement device 100C according to the present embodiment as well, the relative positional relationship between the photoelectric sensor 50 and the radial artery is constantly held during the measurement operation with the detection unit for blood pressure information measurement device having a configuration similar to the cuff 10A described in the first embodiment, whereby the shift in the direction of the photoelectric sensor with respect to the radial artery, which has been a conventional problem, does not arise and a blood pressure information measurement device capable of acquiring the systolic blood pressure value and the diastolic blood pressure value easily and at high accuracy can be realized.

### Fourth Embodiment

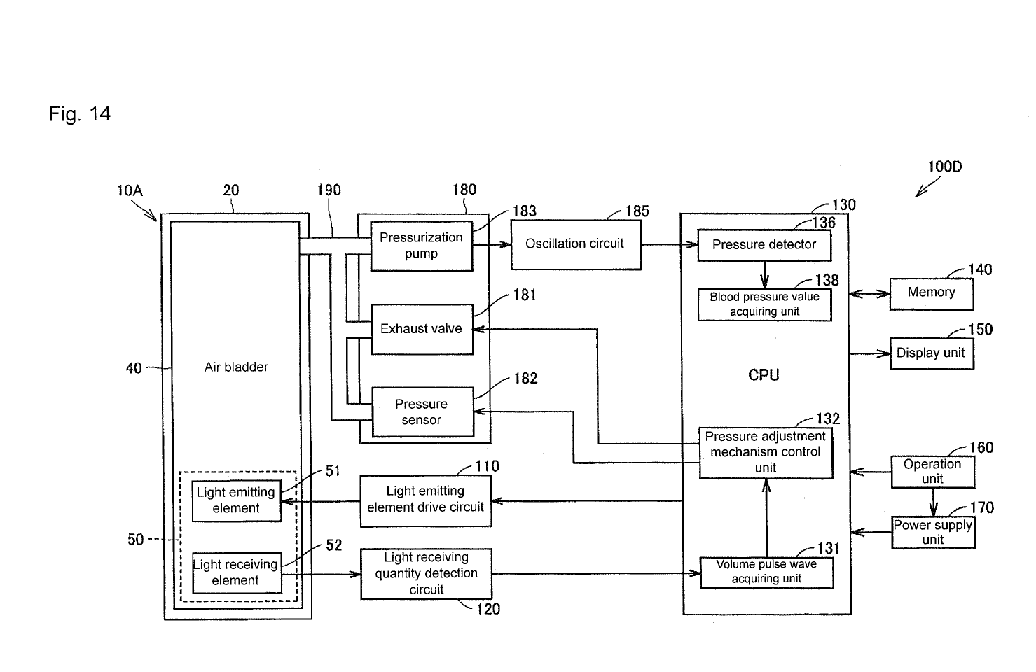

FIG. 14 is a functional block diagram showing a configuration of a blood pressure information measurement device according to a fourth embodiment of the present invention. A configuration of a blood pressure information measurement device 100D according to the present embodiment will be described with reference to FIG. 14. The same reference numerals are denoted for the portions similar to the blood pressure information measurement device 100A in the first embodiment, and the description thereof will not be repeated herein.

The blood pressure information measurement device 100D according to the present embodiment is a blood pressure information measurement device including a blood pressure value acquiring function using a volume compensation method. As shown in FIG. 14, in the blood pressure information measurement device 100D according to the present embodiment, a blood pressure value acquiring unit 138 is arranged in the CPU 130. The blood pressure value acquiring unit 138 acquires the systolic blood pressure value and the diastolic blood pressure value based on the cuff pressure information obtained by the pressure detector 136.

The volume compensation method servo controls the cuff pressure so as to constantly achieve equilibrium between the inner pressure exerted on the blood vessel wall of the artery (pressure generated by the pumping function of the heart, that is, blood pressure) and the external pressure (compression force by the cuff), and acquires the systolic blood pressure value and the diastolic blood pressure value by detecting the cuff pressure at that time.

The blood pressure information measurement device 100D according to the present embodiment includes the air system component 180 having a configuration similar to the blood pressure information measurement device 100A according to the first embodiment, and uses the air system component 180 to perform the servo control of the cuff pressure. The photoelectric sensor 50 arranged in the cuff 10A is used for the setting of the target value of the servo control in this case, and the determination on whether the inner pressure exerted on the blood vessel wall by the servo control and the external pressure are in an equilibrium state.

As opposed to the blood pressure information measurement device 100C including the blood pressure value acquiring function of volume vibration method according to the third embodiment, in the blood pressure information measurement device 100D according to the present embodiment, the pressure adjustment mechanism control unit 132 performs the servo control of the cuff pressure based on the volume blood pressure information acquired by the volume pulse wave acquiring unit 131. The systolic blood pressure value and the diastolic blood pressure value are acquired based on the cuff pressure information obtained by the pressure sensor 183.

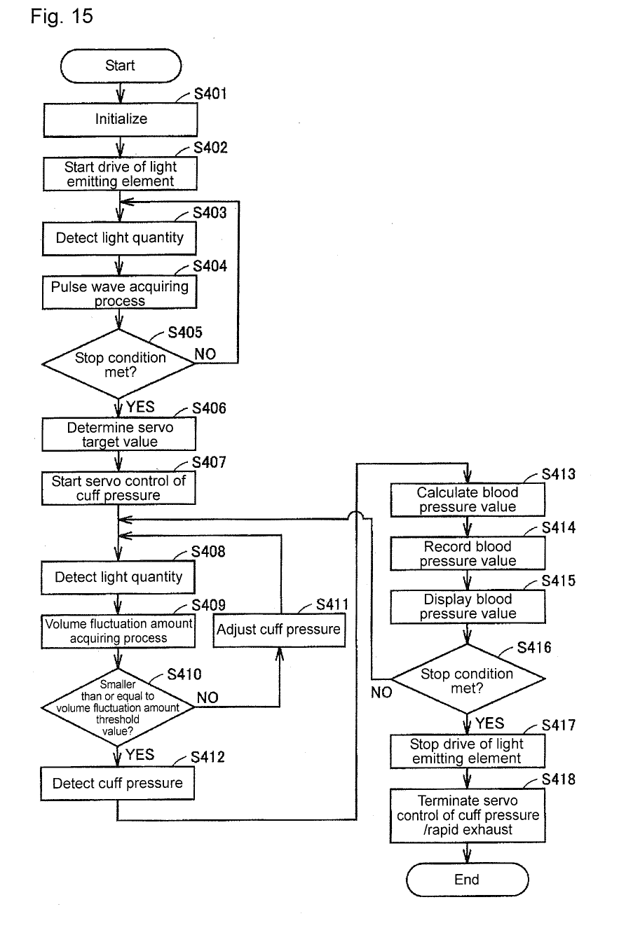

FIG. 15 is a flowchart showing the processing procedures of the blood pressure information measurement device according to the present embodiment. The processing procedures of the blood pressure information measurement device 100D according to the present embodiment will be described below with reference to FIG. 15. The program according to the flowchart is stored in advance in the memory 140 shown in FIG. 14, and the process is implemented when the CPU 130 reads out the program from the memory 140 and executes the same.

As shown in FIG. 15, when the subject operates the operation unit 160 of the blood pressure information measurement device 100D and inputs a command to turn ON power, power as power supply is supplied from the power supply unit 170 to the CPU 130, so that the CPU 130 is driven and initialization of the blood pressure information measurement device 100D is performed (step S401). The subject attaches the cuff 10A serving as the detection unit for blood pressure information measurement device to the wrist in advance.

The CPU 130 then starts to drive the light emitting element 51 through the light emitting element drive circuit 110 (step S402). The detection light is radiated from the light emitting element 51 toward the measuring site including the radial artery. In parallel with the drive of the light emitting element 51, the light receiving quantity detection circuit 120 generates a voltage signal digitized based on the signal inputted from the light receiving element 52 (step S403), and inputs the same to the CPU 130. The CPU 130 acquires the volume pulse wave in the volume pulse wave acquiring unit 131 based on the inputted voltage signal (step S404).

The series of operations including step S403 and step S404 are repeatedly performed until a predetermined stop condition (e.g., when cuff pressure reaches a predetermined level, elapse of a set time by a timer circuit, and the like) is met (NO in step S405). When the predetermined stop condition is met (YES in step S405), the CPU 130 determines the servo target value and the initial control target value of the cuff pressure based on the information of the measured volume pulse wave (S406).

The pressurization pump 181 is then driven by the pressure adjustment mechanism control unit 132 arranged in the CPU 130, the air is sent to the air bladder 40, and the servo control of the cuff pressure is started (step S407). When the cuff pressure reaches the initial control target value, the CPU 130 acquires the volume pulse wave in the volume pulse wave acquiring unit 131 based on the inputted voltage signal (steps S408, S409). Thereafter, whether or not the acquired volume fluctuation amount is smaller than or equal to a predefined threshold value is determined in step S410, and if not determined that the volume fluctuation amount is smaller than or equal to the threshold value (NO in step S410), the cuff pressure adjustment (change of servo target value, servo control of the cuff pressure toward the servo target value after change, and the like) is performed based on the arterial volume signal derived therefrom (step S411), and then the process returns to step S408 to repeat the light quantity detection (step S408), the acquisition of the volume fluctuation amount (step S409) based thereon, and the determination on whether or not the volume fluctuation amount is smaller than or equal to the threshold value (step S410). If determined that the volume fluctuation amount is smaller than or equal to the threshold value defined in advance (YES in step S410), the process proceeds to step S412 to detect the cuff pressure by the pressure sensor 183, and the information thereof is inputted to the pressure detector 136 of the CPU 130 through the oscillation circuit 185.

The cuff pressure information obtained in step S412 is then inputted to the blood pressure value acquiring unit 138 to acquire the systolic blood pressure value and the diastolic blood pressure value (step S413). Thereafter, the systolic blood pressure value and the diastolic blood pressure value acquired by the blood pressure value acquiring unit 138 are stored in the memory 140 as the measurement result (step S414), and the measurement result is displayed by the display unit 150 (step S415). The display unit 150 displays the systolic blood pressure value and the diastolic blood pressure value as numerical values or a graph of the temporal change of values.

The series of operations including step S408 to step S415 are repeatedly performed until a predetermined stop condition (e.g., input of command to stop measurement by the subject, elapse of a set time by a timer circuit, and the like) is met (NO in step S416). When the predetermined stop condition is met (YES in step S416), an instruction to cancel the drive of the light emitting element 51 is made to the light emitting element drive circuit 110 (step S417).

Thereafter, the CPU 103 outputs a command for rapid exhaust with respect to the air system component 180 to stop the servo control of the cuff pressure, and releases the compression of the radial artery (step S418). The blood pressure information measurement device 100D is then in a standby state, and stops the supply of power as the power supply after waiting for the input of command to turn OFF the power by the operation unit 160 of the subject.

The blood pressure information measurement device 100D described above may be a blood pressure information measurement device capable of measuring the systolic blood pressure value and the diastolic blood pressure value. In the blood pressure information measurement device 100D according to the present embodiment as well, the relative positional relationship between the photoelectric sensor 50 and the radial artery is constantly held during the measurement operation with the detection unit for blood pressure information measurement device having a configuration similar to the cuff 10A described in the first embodiment, whereby the shift in the direction of the photoelectric sensor with respect to the radial artery, which has been a conventional problem, does not arise and a blood pressure information measurement device capable of acquiring the systolic blood pressure value and the diastolic blood pressure value easily and at high accuracy can be realized.

In the first to fourth embodiments described above, there has been illustrated and described the detection unit for blood pressure information measurement device configured such that the distal end of the guide portion arranged in the fixing stand is in direct contact with the body surface near the measuring site in the attached state, but the detection unit for blood pressure information measurement device may not necessarily be configured such that the distal end of the guide portion is in direct contact with the body surface, and may be indirectly set on the body surface. The configuration in which the distal end of the guide portion is indirectly set on the body surface includes a configuration in which the fixing stand and the belt member are covered with a cover serving as an outer package body.

In the first to fourth embodiments described above, the blood pressure information measurement device configured to be able to measure the systolic blood pressure value, the diastolic blood pressure value, the pulse wave, the AI value, and the like has been illustrated and described, but the present invention can also be applied to the blood pressure information measurement device capable of measuring a pulse beat, an average blood pressure value, and the like.

Furthermore, in the first to fourth embodiments, a case of adopting the wrist as the measuring site has been illustrated and described, but the present invention may also be obviously applied to the blood pressure information measurement device in which other sites of the body are adopted as the measuring site. The other sites of the body that may be adopted as the measuring site include other sites of four limbs such as an upper arm, an ankle, and a thigh, a neck, a finger, and the like.

Each embodiment disclosed herein is illustrative in all aspects, and should not be construed as being restrictive. The technical scope of the present invention is defined by the claims, and meanings equivalent with the description of the claims and all modifications within the scope are to be encompassed herein.

1. A detection unit for blood pressure information measurement device comprising:
a compression fluid bag for compressing an artery in a measuring site by compressing the measuring site;
a photoelectric sensor, including a light emitting portion and a light receiving portion for radiating detection light toward the measuring site from the light emitting portion, receiving the detection light transmitted through the measuring site with the light receiving portion, and outputting an output signal corresponding to a light quantity of the received detection light; and
a fixing unit for fixing the photoelectric sensor with respect to the measuring site; wherein
the fixing unit includes a base portion with a sensor attachment surface attached with the photoelectric sensor, and a guide portion arranged projecting out from the base portion toward the sensor attachment surface side, the guide portion having a distal end directly or indirectly placed to a body surface near the measuring site with the photoelectric sensor fixed with respect to the measuring site by the fixing unit;
the compression fluid bag is arranged on the sensor attachment surface so as to cover the photoelectric sensor; and
the guide portion is positioned to surround the photoelectric sensor when the fixing unit is seen from a normal direction of the sensor attachment surface.

2. The detection unit for blood pressure information measurement device according to claim 1, wherein the guide portion has a wall-shape or a columnar shape.

3. The detection unit for blood pressure information measurement device according to claim 1, wherein the fixing unit includes a belt member attached by being wrapped around a living body including the measuring site.

4. The detection unit for blood pressure information measurement device according to claim 3, wherein the light emitting portion and the light receiving portion are arranged in line in a longitudinal direction of the belt member.

5. A blood pressure information measurement device comprising:
the detection unit for blood pressure information device according to claim 1;
a drive unit for causing the light emitting portion to emit light;
a light receiving quantity detector for detecting a fluctuation of light receiving quantity based on an output signal outputted from the photoelectric sensor; and
a volume pulse wave acquiring unit for acquiring a volume pulse wave of an artery based on information obtained by the light receiving quantity detector.

6. The blood pressure information measurement device according to claim 5, wherein the drive unit causes the light emitting portion to intermittently emit pulsed light.

7. The blood pressure information measurement device according to claim 5, further comprising a pressure adjustment mechanism for expanding and contracting the compression fluid bag by adjusting an internal pressure of the compression fluid bag.

8. The blood pressure information measurement device according to claim 5, further comprising an ejection wave/reflection wave acquiring unit for acquiring at least one of an ejection wave and a reflection wave of the pulse wave based on information of the volume pulse wave obtained by the volume pulse wave acquiring unit.

9. The blood pressure information measurement device according to claim 5, further comprising:
a compression force detector for detecting an internal pressure of the compression fluid bag; and
a blood pressure value acquiring unit for acquiring a diastolic blood pressure value and a systolic blood pressure value based on information of the volume pulse wave obtained by the volume pulse wave acquiring unit and information of the pressure obtained by the compression force detector.

10. The blood pressure information measurement device according to claim 5, further comprising:
a compression force detector for detecting an internal pressure of the compression fluid bag;
a compression force control unit for servo controlling the compression force with respect to the artery by the compression fluid bag based on information of the volume pulse wave obtained by the volume pulse wave acquiring unit; and
a blood pressure value acquiring unit for acquiring a diastolic blood pressure value and a systolic blood pressure value based on information of the pressure obtained by the compression force detector.
